(19) United States
(12) Patent Application Publication
(10) Pub. No.: US 2010/0249224 A1
(43) Pub. Date: Sep. 30, 2010

(54) Methods and compositions for modulating glutamate dehydrogenase

(76) Inventors: Thomas Smith, St. Louis, MO (US); Charles Stanley, Drexel Hill, PA (US); Franz Matschinsky, Wallingford, PA (US)

Correspondence Address: BRYAN CAVE LLP, 211 NORTH BROADWAY, SUITE 3600, ST. LOUIS, MO, 63102-2750, US

(21) Appl. No.: 12/778,653

(22) Filed: May 12, 2010

Related U.S. Application Data
(63) Continuation of application No. 11/263,797, filed on Nov. 1, 2005, now abandoned.
(60) Provisional application No. 60/624,025, filed on Nov. 1, 2004.

(51) Int. Cl. A61K 31/353; A61P 3/10; A61P 5/48; C12N 9/99

(52) U.S. Cl. 514/456; 435/184

(57) ABSTRACT

Compositions, compounds, and methods for modulating the activity of glutamate dehydrogenase are disclosed. In addition, compositions, compounds, and methods for regulating insulin secretion and treating hyperinsulism/hyperammonemia and/or diabetes are disclosed.

### CROSS-REFERENCE TO RELATED APPLICATIONS

This application is a continuation of U.S. patent application Ser. No. 11/263,797, filed Nov. 1, 2005, which claims benefit under 35 U.S.C. §119(e) to U.S. Provisional Application Ser. No. 60/624,025, filed Nov. 1, 2004.

The work of this invention was supported in part by a grant from the U.S. National Institutes of Health. The United States Government may have certain rights to this invention.

### FIELD OF THE INVENTION

The present invention relates to the field of medicine and medical research. More particularly, the present invention relates to compositions, compounds, and methods for modulating the activity of glutamate dehydrogenase. In addition, in certain embodiments, the invention relates to compositions, compounds, and methods for regulating insulin secretion and treating hyperinsulism/hyperammonemia and/or diabetes.

### BACKGROUND OF THE INVENTION

The mitochondria of the pancreatic β-cell play an integrative role in regulating insulin secretion. In particular, the glutamate dehydrogenase enzyme (also referred to herein as “GDH”) within such mitochondria is believed to affect insulin homeostasis. GDH is known to catalyze the oxidative deamination of L-glutamate and exhibit complex regulation in mammals through inhibition by palmitoyl-coenzyme A (“palmitoyl CoA”), guanosine-5′-triphosphate (“GTP”), and adenosine-5′-triphosphate (“ATP”), and activation by adenosine-5′-diphosphate (“ADP”) and leucine. The connection between GDH and insulin regulation was initially demonstrated using a nonmetabolizable analog of leucine, β-2-aminobicycle[2.2.1]heptane-2-carboxylic acid (“BCH”). Specifically, it has been demonstrated that activation of GDH, e.g., by BCH-induced activation, is tightly correlated with increased glutaminolysis (i.e., the conversion of the amino acid glutamine to lactate), which has been shown to indirectly stimulate insulin secretion.

The in vivo importance of GDH in insulin homeostasis is further demonstrated by the discovery that a genetic disorder, hyperinsulism/hyperammonemia syndrome (referred to herein as “HI/HA”), is caused by dysregulation of GDH. Specifically, it is believed that HI/HA syndrome is caused by impaired (or abrogated) GDH sensitivity to GTP inhibition. More particularly, the GDH enzyme in such HI/HA individuals comprise one or more mutations in its GTP binding site, which are believed to act by sterically interfering with GTP binding. As a result, patients with HI/HA have increased β-cell responsiveness to leucine and susceptibility to hypoglycemia following high protein meals, fasting hypoglycemia, and leucine hypersensitivity.

In light of the foregoing, there is a demand for compositions and methods that may be used to modulate the activity of GDH and, preferably, regulate insulin secretion. In addition, there is a continuing demand for compositions and methods for treating and preventing the effects of disorders relating to the dysregulation of insulin secretion, such as HI/HA. Preferably, the foregoing is achieved through the use of a non-toxic pharmacological agent that allosterically regulates GDH.

### SUMMARY OF THE INVENTION

One embodiment of the present invention is a method for modulating the activity of glutamate dehydrogenase comprising providing to a system in need thereof with an effective amount of a compound selected from the group consisting of EGCG (Epigallocatechin gallate) and ECG (Epicatechin gallate), including salts, hydrates, solvates, N-oxides, structural analogues, and combinations thereof. A further embodiment of the present invention is a method for modulating the activity of glutamate dehydrogenase comprising providing to a system in need thereof with an effective amount of Camellia sinensis extract.

Another embodiment of the present invention is a method for regulating insulin secretion comprising providing to a patient in need thereof with an effective amount of a composition selected from the group consisting of Camellia sinensis extract, EGCG, and ECG, including salts, hydrates, solvates, N-oxides, structural analogues, and combinations thereof.

A further embodiment of the present invention is a method for treating or preventing the effects of disorders relating to the dysregulation of insulin secretion, such as HI/HA, wherein such method comprises providing to a patient in need thereof with an effective amount of a compound selected from the group consisting of EGCG and ECG, including salts, hydrates, solvates, N-oxides, structural analogues, and combinations thereof.

Another embodiment of the present invention is a method for treating or preventing the effects of HI/HA, which comprises providing to a patient in need thereof with an effective amount of Camellia sinensis extract.

A still further embodiment of the present invention is a composition for treating or preventing the effects of HI/HA, which comprises an effective amount of a compound selected from the group consisting of EGCG and ECG, including salts, hydrates, solvates, N-oxides, structural analogues, and combinations thereof, and an appropriate carrier.

The above-mentioned and additional features of the present invention are further illustrated in the Detailed Description contained herein. All references disclosed herein, including U.S. patents and published patent applications, are hereby incorporated by reference in their entirety as if each was incorporated individually.

### BRIEF DESCRIPTION OF THE FIGURES

FIG. 1. The effects of the polyphenols EGCG, ECG, EGO, and EC (defined herein), from Camellia sinensis, on bovine glutamate dehydrogenase activity. (A) A line graph showing dose response curves and the effects of such polyphenols on the reductive amination reaction catalyzed by bovine glutamate dehydrogenase. (B) The chemical structures of the various polyphenol compounds tested.

FIG. 2. Effects of EGCG on glutamate dehydrogenase steady state reaction. (A) A Lineweaver-Burke plot of the reductive amination reaction in the presence of varying concentrations of 2-oxoglutarate (α-ketoglutarate). “αKG” refers to α-ketoglutarate. (B) A Lineweaver-Burke plot of the reductive amination reaction in the presence of varying concentrations of NADH (nicotinamide adenine dinucleotide).

FIG. 3. Abrogation of EGCG inhibition by leucine, BCH, and ADP. The percent activity for each curve shown in FIG. 3 is relative to the velocity of the reaction in the absence of activator (i.e., leucine, BCH, or ADP). (A) A line graph showing the reversal of EGCG inhibition by leucine and BCH. The grey lines represent the reaction at varied leucine concentrations in the presence and absence of EGCG, whereas the black lines represent the change in velocity at varied BCH concentrations. (B) A line graph showing the reversal of EGCG inhibition by ADP.

FIG. 4. A line graph showing EGCG dose-response curves and inhibition of EGCG on the activity of various forms of GDH.

FIG. 5. A line graph showing the effects of EGCG and EGO on BCH-stimulated insulin secretion.

FIG. 6. Effects of EGCG and EGO on BCH-stimulated insulin secretion and oxygen consumption. (A) A line graph showing the effect of EGCG and EGO on leucine (or BCH)-stimulated insulin secretion (open diamonds: 0 μM EGCG; solid diamonds: 20 μM EGCG; grey circles: 20 μM EGO). (B) A line graph showing the effect of EGCG and EGO on leucine (or BCH)-stimulated oxygen consumption (light grey line: 0 μM EGCG; black line: 20 μM EGCG; dark grey line: 20 μM EGO 20).

FIG. 7. Effects of EGCG and 6-diazo-5-oxo-L-norleucine (“DON”) on [U-¹⁴C]-glutamine oxidation. (A) A line graph showing BCH dose-dependently stimulated glutamine oxidation for 100 islets from 2 mM glutamine (open triangles: BCH only; solid squares: BCH and 20 μM EGCG; solid circle: BCH and 20 μM EGO). (B) and (C) Line graphs showing EGCG and DON dose-dependently inhibited 10 mM BCH-stimulated glutamine oxidation for 100 islets from 3 mM glutamine (dashed line shows rate of glutamine oxidation from 3 mM glutamine only—no BCH added). (D) A bar graph showing the effects of BCH, EGCG and DON on glutamine oxidation from 3 mM glutamine.

FIG. 8. A line graph showing the effects of EGCG on glucose stimulated insulin secretion.

FIG. 9. Effects of EGCG on glucose stimulated insulin secretion and oxygen consumption. (A) A line graph showing insulin secretion in response to the various conditions described and shown herein (open diamonds: 0 μM EGCG; solid diamonds: 20 μM EGCG; grey circles: 20 μM EGO). (B) A line graph showing oxygen consumption in response to the various conditions described and shown herein (light grey: 0 μM EGCG; black: 20 μM EGCG; darker grey: 20 μM EGC). As used herein, “G” refers to glucose and “FCCP” refers to carbonyl cyanide 4-(trifluoromethoxy)phenylhydrazone (a.k.a. Mesoxalonitrile 4-trifluoromethoxyphenylhydrazone).

FIG. 10. Effects of EGCG on [U-¹⁴C]-glucose oxidation. A line graph showing the effects of EGCG on glucose oxidation in a dose-dependent manner, wherein a 3 mM dose is represented by solid circles and a 12 mM dose is represented by solid triangles.

FIG. 11. Effects of EGCG in glucose stimulated insulin secretion in 120 minute “run-down” islets.

FIG. 12. Model for EGCG effects on GSIS (glucose stimulated insulin secretion) over long “run-down” conditions. The lower panel shows the effects of EGCG on GSIS when the intracellular levels of ATP and GTP have been depleted by longer “run-down” times. EGCG inhibits GDH activity and preserves a larger pool of glutamate, thereby increasing the concentration of glutamine that is then able to potentiate the β-cell response to the increased ATP levels derived from glucose metabolism.

### DETAILED DESCRIPTION OF THE INVENTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used, and made without departing from the scope and spirit of the invention.

It is well known that the Camellia sinensis plant (a.k.a. Green tea) is a significant source of a certain type of flavonoids referred to as catechins. Such catechins include epigallocatechin gallate (EGCG), epigallocatechin (EGO), epicatechin gallate (ECG), and epicatechin (EC). Over the past several decades, there has been growing interest in EGCG, as it has been suggested to, among other things, decrease cholesterol levels, act as an antibiotic and anticarcinogen, and repress hepatic glucose production. The exact mechanism of action of EGCG, with regard to these various effects, is largely unknown and in many cases is assumed to be the result of its apparent antioxidant activity.

In a first embodiment of the present invention, methods for modulating the activity of glutamate dehydrogenase are provided, which comprise providing to a system in need thereof with an effective amount of a compound selected from the group consisting of EGCG, ECG, and combinations thereof. As used herein, “system” may be, without limitation, an experimental system, including an in vitro and/or in vivo system, or a mammal, such as a human patient or veterinarian patient, which is provided one or more compositions described herein to be useful in practicing the invention.

As used herein, “EGCG” refers to Epigallocatechin gallate (a.k.a. (2R,3R)-2-(3,4,5-Trihydroxyphenyl)-3,4-dihydro-1(2H)-benzopyran-3,5,7-triol 3-(3,4,5-trihydroxybenzoate), including salts, hydrates, solvates, structural analogues, and N-oxides thereof. In certain embodiments, EGCG has a chemical formula of C₂₂H₁₈O₁₁ and a molecular weight of approximately 458.37. The chemical structure of a non-limiting example of EGCG is shown below:

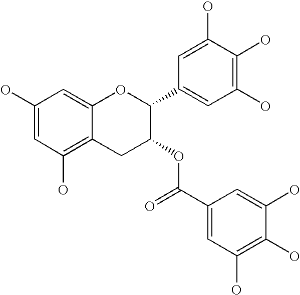

As used herein, “ECG” refers to Epicatechin gallate (a.k.a. (2R,3R)-2-(3,4-Dihydroxyphenyl)-3,4-dihydro-1(2H)-benzopyran-3,5,7-triol 3-(3,4,5-trihydroxybenzoate)), including salts, hydrates, solvates, structural analogues, and N-oxides thereof. In certain embodiments, ECG has a chemical formula of C₂₂H₁₈O₁₀ and a molecular weight of approximately 442.37. The chemical structure of a non-limiting example of ECG is shown below:

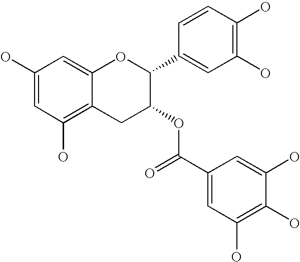

The EGCG and ECG compounds described herein contain one or more asymmetric centers and thus give rise to enantiomers, diastereomers, and other stereoisomeric forms that may be defined, in terms of absolute stereochemistry, as (R)- or (S)-. The present invention encompasses all such possible isomers, as well as their racemic and optically pure forms. Optical isomers may be prepared from their respective optically active precursors, or by resolving the racemic mixtures. Such resolution may be carried out in the presence of a resolving agent, by chromatography, or by repeated crystallization or by some combination of such techniques which are known to those skilled in the art.

In addition, when the compounds described herein contain olefinic double bonds, other unsaturation, or other centers of geometric asymmetry, and unless specified otherwise, it is intended that the compounds include both E and Z geometric isomers or cis- and trans-isomers. Similarly, all tautomeric forms are intended to be encompassed by the present invention. The configuration of any carbon-carbon double bond appearing herein is selected for convenience only and is not intended to designate a particular configuration unless the text herein so states; thus, a carbon-carbon double bond or carbon-heteroatom double bond depicted arbitrarily herein as trans may be cis, trans, or a mixture of the two in any proportion.

In certain preferred embodiments, the EGCG and/or ECG compounds are in a substantially isolated and purified form. As used herein, “substantially isolated and purified form” means that the EGCG or ECG compound, for example, is separated from its native environment in sufficiently pure form so that it can be manipulated or used for any desired purpose. For example, in certain embodiments, such compound (alone or in combination with other GDH-modulating compounds) may constitute at least 40% (wt) of the total composition used to practice the claimed invention, or preferably at least 60% (wt), or more preferably at least 80% (wt), or still more preferably at least 90% (wt).

Still further, the EGCG and ECG compounds disclosed herein may be modified by appending any desired functionalities to enhance selective biological properties. Such modifications are known in the art and may include those which increase the activity, bioavailability, biological penetration into a given system or substrate, solubility, half-life, or other desirable characteristic of such EGCG and/or ECG compound. In addition, such modifications may reduce the relative toxicity of such EGCG and/or ECG compound.

Synthetic chemistry transformations and protecting group methodologies (protection and deprotection) useful in synthesizing and/or derivatizing the EGCG and/or ECG compounds described herein are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Accordingly, as used herein, EGCG and ECG encompass variations of the above-mentioned chemical formulas and structures. For example, the invention provides that EGCG includes the following compound:

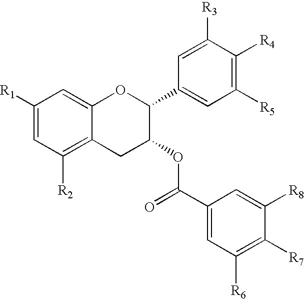

, wherein R₁ through R₈ are independently selected from the group consisting of halogen, hydrogen, oxygen, hydroxy, carbonyl, and other functional groups known in the art that provide desirable characteristics to the compound.

Similarly, the invention provides that ECG includes the following compound:

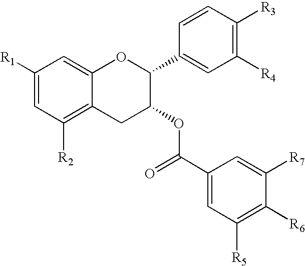

, wherein R₁ through R₇ are independently selected from the group consisting of halogen, hydrogen, oxygen, hydroxy, carbonyl, and other functional groups known in the art that provide desirable characteristics to the compound.

In certain embodiments of the present invention, the methods for modulating the activity of glutamate dehydrogenase comprise providing to an in vitro system an effective amount of Camellia sinensis extract, EGCG, ECG, or combinations thereof. In certain embodiments, the in vitro system, optionally, includes purified glutamate dehydrogenase. As used herein, “purified glutamate dehydrogenase” means that the glutamate dehydrogenase enzyme has been separated from its native environment in sufficiently pure form so that it can be manipulated or used for any desired purpose. For example, such enzyme may be sufficiently pure to be used to catalyze the oxidative deamination of L-glutamate (in the presence of the necessary substrates, co-factors, enzymes, and other molecules under suitable conditions). In other preferred embodiments, the Camellia sinensis extract, EGCG and/or ECG compositions may be provided to an in vitro system that comprises glutamate dehydrogenase in its natural environment, such as within a particular cell line and/or tissue type, e.g., within isolated pancreatic β-cells.

In other preferred embodiments, the methods for modulating the activity of glutamate dehydrogenase comprise providing to a patient in need thereof with an effective amount of Camellia sinensis extract, EGCG, ECG, or combinations thereof. In such embodiments, for example, the Camellia sinensis extract, EGCG and/or ECG compositions may be provided to a patient to (i) regulate insulin secretion, such as to regulate leucine stimulated insulin secretion, (ii) treat and/or prevent the effects of HI/HA, and/or (iii) treat and/or prevent the effects of diabetes. In certain embodiments of the present invention, the methods for modulating the activity of glutamate dehydrogenase comprise providing to a system in need thereof with an effective amount of Camellia sinensis extract, which, in certain preferred embodiments, is an aqueous Camellia sinensis extract. Those of ordinary skill in the art will appreciate, however, that such Camellia sinensis extract may be aqueous or non-aqueous. For example, the Camellia sinensis extract may be formulated in connection with any aqueous or non-aqueous pharmaceutically acceptable carrier, such as those carriers described herein.

Non-limiting examples of EGCG and ECG compositions may be found in relatively high levels within Camellia sinensis extract. While Camellia sinensis extracts, and EGCG and ECG compositions derived therefrom, may be used in certain preferred embodiments of the present invention, those of ordinary skill will appreciate that other sources of EGCG and ECG compositions may be used. For example, the invention provides that extracts from other fruits may be used, such as grape, apple, apricot, blackberry, or cherry or products from scutellaria and bamboo. Furthermore, such alternative sources may be used to isolate and purify EGCG and ECG compositions using methods well known in the art.

The EGCG and/or ECG compositions described herein may be isolated and purified from natural sources and, optionally, modified as described herein. For example, EGCG and/or ECG compositions that may be used in practicing the present invention may be extracted from Camellia sinensis. A non-limiting example of a protocol that may be used to isolate and purify EGCG and/or ECG compositions is described in U.S. patent application publication 2005/0176939, which is hereby incorporated by reference in its entirety. In addition, methods for producing Camellia sinensis extracts with high EGCG ratios, for example, were reported in Copland et al., 1998. Food Chem. 61: 81-87). Similarly, the use of high-speed counter-current chromatography as a fractionation tool for both crude extracts and semi-purified fractions, as well as for the production of purified catechin, from crude Camellia sinensis extracts was reported in Du et al., 1997. res. Develop. Basic Agric. and High Technol., 1: 40-47 and Du et al., 1998. J. Liq. Chromatog. & Related Technol., 21: 203-208.

Alternatively, such EGCG and ECG compositions may be purchased from commercial vendors. In many cases, such EGCG and ECG compositions are commercially-available in substantially purified forms. For example, EGCG and ECG compositions are offered by Sigma-Aldrich Chemical Company (St. Louis, Mo.). After isolating and purifying such EGCG and ECG compositions from natural sources (or otherwise obtaining such compositions from commercial vendors), the EGCG and ECG compositions may be diluted and/or solubilized in any suitable solvent.

EGCG (and ECG) are soluble in, for example, organic solvents such as ethanol, dimethyl sulfoxide (DMSO), and dimethyl formamide. The solubility of EGCG, for example, in these solvents is at least 20 mg/mL. EGCG has been shown to be stable for at least six months in these solvents if stored at −20° C. Preferably, further dilutions of stock solutions comprising EGCG and/or ECG into aqueous buffers, or isotonic saline, should be made prior to, for example, performing biological experiments or providing such compositions to a patient. In certain embodiments of the present invention, it is generally preferred that any residual amount of such organic solvent is insignificant, since organic solvents may have undesirable physiological effects at sufficient concentrations. Organic solvent-free aqueous solutions of EGCG, for example, in certain embodiments, may be prepared by directly dissolving the crystalline compound in aqueous buffers. The solubility of EGCG in phosphate buffered saline (PBS) (pH 7.2), for example, is at least 25 mg/mL.

Those of ordinary skill in the art will appreciate that the EGCG and ECG compositions of the present invention may be further modified, purified, and/or combined with other agents after such compositions are isolated and purified from a natural source, such as Camellia sinensis, or otherwise purchased from a vendor.

As will be shown and demonstrated in the Examples below, the inventors have discovered that EGCG (and ECG) allosterically modulate GDH activity. More particularly, the inventors have discovered that EGCG (and ECG) allosterically inhibit GDH activity. Such inhibition was demonstrated, in-vitro, with a nanomolar ED₅₀. As described and shown herein, because GDH activity is inhibited by the presence of EGCG and ECG, but not EC or EGO, such inhibition cannot be due to the antioxidant properties of such compositions (as EC and EGC are known to exhibit substantially similar antioxidant properties as EGCG and ECG).

Indeed, as described and shown herein, EGCG and ECG inhibition of GDH activity is non-competitive and, similar to GTP inhibition, is abrogated by leucine, BCH, and ADP. Importantly, as described and shown herein, the GDH enzyme found in HI/HA patients, as well as the GDH enzyme from Tetrahymena thermophilia (“tGDH”), are all inhibited by EGCG. It is well known that such HI/HA GDH and tGDH enzymes have dysfunctional GTP binding sites. Accordingly, it is unlikely that EGCG and ECG act by binding to the GTP site of GDH.

In addition, as described and shown herein, the specificity of EGCG (and ECG) for GDH inhibition is observed in pancreatic β-cells. The specificity of such inhibition was confirmed by demonstrating that EGCG, but not epigallocatechin (EGO), causes a concomitant blockade of glutaminolysis stimulated by BCH, but not in the basal level of glutamine oxidation or cellular respiration. Still further, as demonstrated below, when EGCG, for example, is added to pancreatic β-cells during glucose stimulation under conditions that GDH does not play a major role in the regulation of insulin secretion, no effect is observed on insulin secretion, glucose oxidation, or cellular respiration.

HI/HA syndrome has been shown to be caused by impaired (or abrogated) GDH sensitivity to GTP inhibition. More particularly, the GDH enzyme in HI/HA individuals comprise one or more mutations in its GTP binding site, which are believed to act by sterically interfering with GTP binding. As a result, patients with HI/HA have increased β-cell responsiveness to leucine and susceptibility to hypoglycemia following high protein meals, fasting hypoglycemia, and leucine hypersensitivity.

In light of the foregoing, certain preferred embodiments of the present invention provide methods for treating or preventing the effects of HI/HA comprising providing to a patient in need thereof with an effective amount of a compound selected from the group consisting of EGCG and ECG, including salts, hydrates, solvates, N-oxides, structural analogues, and combinations thereof. The invention provides that such patient may be of any age. In certain preferred embodiments, however, the patient is a pediatric patient.

Similarly, further embodiments of the present invention provide methods for treating or preventing the effects of HI/HA comprising providing to a patient in need thereof with an effective amount of Camellia sinensis extract. The methods and compositions described herein to be useful for “treating or preventing the effects of HI/HA” may, for example, (i) inhibit or reduce the activity of GDH in such individuals or (ii) reduce the extent to which such individuals are susceptible to hypoglycemia following high protein meals, fasting hypoglycemia, and/or leucine hypersensitivity.

Still further embodiments of the present invention provide methods for regulating insulin secretion, which comprise providing to a patient in need thereof with an effective amount of a composition selected from the group consisting of Camellia sinensis extract, EGCG, and ECG, including salts, hydrates, solvates, N-oxides, structural analogues, and combinations thereof.

As described and demonstrated herein, in certain embodiments, the present invention provides that Camellia sinensis extract, EGCG, and/or ECG may be used to modulate GDH activity and insulin secretion under certain conditions. For example, such compositions may be used to regulate leucine stimulated insulin secretion (LSIS) by inhibiting GDH activity. Still further, for example, such compositions may be used to regulate LSIS (by inhibiting GDH activity), which may occur after an individual consumes a high protein meal. In addition, such methods for modulating GDH activity and/or insulin secretion may include a monitoring step. For example, for medical applications to treat an insulin-related disorder, the invention provides that the insulin level may be measured in a patient, such as using any immunoassay (e.g., radioimmunoassays). Next, the insulin level measured in such patient may be compared to a preferred range, which those skilled in the art will appreciate may depend on, among other things, the age, weight, height, and gender of the patient. If the insulin level is outside of the preferred range, the patient may be provided with an effective amount of EGCG, ECG, Camellia sinensis extract, or a combination thereof.

In certain preferred embodiments of the present invention, compositions are provided for treating and/or preventing the effects of a disorder caused by irregular (or the dysregulation of) insulin secretion, such as HI/HA and/or diabetes. In such embodiments, the compositions preferably comprise an effective amount of a compound selected from the group consisting of EGCG and ECG, including salts, hydrates, solvates, N-oxides, structural analogues, and combinations thereof, and an appropriate carrier.

The compositions useful in the present invention may, optionally, be converted to their therapeutically-active non-toxic acid salt forms by treatment with appropriate acids. Such acids include inorganic acids, e.g., hydrochloric and hydrobromic acids, sulfuric acid, nitric acid, phosphoric acid and like acids; or organic acids, such as acetic, propanoic, hydroxyacetic, 2-hydroxypropanoic, 2-oxo-propanoic, ethanedioic, propanedioic and like acids. Of course, the salt forms may be converted into the free base form by treatment with alkali. The pharmaceutically-acceptable acid salts of the present invention also comprise the solvates that the compositions of the present invention may form, which, of course, are included within the scope of the present invention. Non-limiting examples of such solvates are hydrates, alcoholates and the like.

Such pharmacologic compositions may be formulated in various ways known in the art for administration purposes. To prepare the pharmaceutical compositions of the present invention, an effective amount of the particular compound, in base or acid salt form, as the active ingredient is combined with one or more pharmaceutically-acceptable carriers and delivery vehicles. As discussed herein, numerous pharmaceutically acceptable carriers and delivery vehicles exist that are readily accessible and well-known in the art, which may be employed to generate the preparation desired. Representative examples of pharmaceutically acceptable carriers and delivery vehicles include aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene-polyoxypropylene-block polymers, polyethylene glycol and wool fat, and the like.

The pharmacologic compositions described herein may further be prepared in unitary dosage form suitable for administration orally, percutaneously, by parenteral injection (including subcutaneous, intramuscular, intravenous and intradermal), topically, or for application to a medical device, such as an implant, catheter, or other device.

In preparing the compositions for oral dosage, for example, any of the pharmaceutical media known in the art may be used, such as water, glycols, oils, alcohols and the like in the case of oral liquid preparations such as suspensions, syrups, elixirs and solutions. When solid carriers are desired, starches, sugars, kaolin, lubricants, binders, cellulose and its derivatives, and disintegrating agents and the like may be used to prepare, for example, powders, pills, capsules and tablets.

For parenteral compositions, acceptable carriers often comprise sterile water, which may be supplemented with various solutes to, for example, increase solubility. Injectable solutions may be prepared in which the carrier comprises saline solution, glucose solution, or a mixture thereof, which may include certain well-known anti-oxidants, buffers, bacteriostats, and other solutes that render the formulation isotonic with the blood of the intended patient.

For percutaneous administration, the carrier may, optionally, comprise a penetration enhancing agent and/or a suitable wetting agent. Dosage forms for topical or transdermal administration of a compound of this invention include ointments, pastes, creams, lotions, gels, powders, solutions, sprays, inhalants or patches. The active compound is mixed under sterile conditions with a pharmaceutically acceptable carrier and optionally one or more preservatives and/or buffers. The ointments, pastes, creams and gels may contain, in addition to an active compound according to the present invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In some cases, the pH of the pharmaceutical formulations contemplated herein may be adjusted with acceptable acids, bases or buffers to enhance the stability of the active compound or its delivery form.

Still further, in order to prolong the activity of, for example, an EGCG and/or ECG composition disclosed herein, it may be desirable to slow the absorption of the composition from subcutaneous or intramuscular injection. This may be accomplished using a liquid suspension of crystalline or amorphous material with poor water solubility. The rate of absorption of the compound then depends upon its rate of dissolution, which, in turn, may depend upon crystal size and crystalline form. Alternatively, delayed absorption of a parenterally administered drug form may be accomplished by dissolving or suspending the compound in an oil vehicle.

Injectable depot forms are made, e.g., by forming microencapsule matrices of one or more compounds of the present invention in biodegradable polymers such as polylactide-polyglycolide. Depending upon the ratio of active compound, e.g., EGCG and/or ECG, to polymer and the nature of the particular polymer employed, the rate at which such compound(s) is released may be controlled. Examples of other such polymers include poly(orthoesters), poly(anhydrides), polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

Depot injectable formulations are also prepared by entrapping the compound in liposomes or microemulsions that are compatible with body tissues.

The compositions of the present invention may also be coupled with soluble polymers as targetable drug carriers. Such polymers may include, for example, polyvinylpyrrolidone, pyran copolymer, polyhydroxypropylmethacrylamide phenyl, polyhydroxyethylaspartamide-phenol, or polyethyleneoxide-polylysine substituted with palmitoyl residues.

In certain embodiments, the compositions described herein may be provided to a patient before, during, or after a HI/HA symptom has formed (or other symptom caused by a disorder relating to irregular insulin secretion, such as diabetes). Thus, such compositions may be administered after a patient has developed, for example, a HI/HA symptom or as a prophylactic to prevent the occurrence (or re-occurrence) of a HI/HA symptom.

The compositions and methods of the present invention may be used to modulate the activity of GDH, regulate insulin secretion, treat or prevent the effects of HI/HA and/or other disorders relating to or caused by dysfunctional regulation of insulin secretion, such as diabetes and similar disorders. In the case of medical applications, for example, the methods of the present invention comprise the steps of providing an effective amount of at least one composition described herein to a patient or, in the case of veterinary applications, to an animal. While the following description makes reference to specific methods and uses of the disclosed compositions for human applications, it should be appreciated that such compositions and methods may be equally useful in animals and, particularly, in veterinary applications.

According to the methods of using the EGCG, ECG, and Camellia sinensis extract compositions disclosed herein for human patient applications, such compositions are, preferably, provided to patients by administering or providing a therapeutically effective amount of such composition, in such amounts and for such time as is necessary to achieve the desired result.

As used herein, a therapeutically “effective amount” of a composition is an amount sufficient to inhibit, reduce, or otherwise modulate the activity of GDH in the system or patient; inhibit, reduce, or otherwise modulate the secretion of insulin in the system or patient; or effectively treat, control and/or prevent the symptoms, or other physiological or biochemical causes or effects, associated with HI/HA (and/or other disorders relating to or caused by dysfunctional regulation of insulin secretion, such as diabetes and similar disorders).

The specific therapeutically effective dose level for any particular patient may depend upon a variety of factors, including the extent to which it is desired to modulate GDH activity, regulate insulin secretion, and/or treat an insulin-related disorder, such as HI/HA or diabetes. In addition, the activity of the specific composition employed; the specific pharmacologic formulation employed; the age, body weight, general health, gender and diet of the patient; the time of administration, route of administration, and rate of excretion of the specific compound employed; the duration of the treatment; drugs used in combination or contemporaneously with the specific compound employed; and like factors well known in the medical arts will influence the specific therapeutically effective dose level. Furthermore, it may be appropriate to administer the required dose more than once in a twenty-four hour period, such as in two, three, four or more sub-doses at appropriate intervals throughout the day.

By way of example only, the total daily dose of one or more of the GDH-modulating compositions disclosed herein may be provided to a patient in single or in divided doses, which may be in amounts from 0.01 to 50 mg/kg body weight or, more typically, from 0.1 to 25 mg/kg body weight. Single dose compositions may contain such amounts or submultiples thereof to make up the daily dose. More preferably, treatment regimens according to the present invention may comprise administering to a patient about 10 mg to about 1000 mg of the GDH-modulating composition(s) disclosed herein, per day in single or multiple doses.

In still further embodiments of the present invention, similar to other compounds such as 6-diazo-5-oxo-L-norleucine (DON) and BCH, EGCG and ECG provide new pharmacological compositions and methods that may be used in research and development of agents (and methods) that regulate insulin secretion and to otherwise dissect and understand the metabolic pathways that regulate insulin secretion. Of course, diabetic disorders are manifested by dysfunctional insulin secretion regulation and, therefore, the invention provides that EGCG and ECG provide new pharmacological compositions that may be used in the treatment of such diabetic disorders.

Still further, in certain embodiments, the invention provides that the GDH enzyme, and EGCG, ECG, and Camellia sinensis extract, may be used in the research, development, and screening of agents (and methods) that may be used to treat, for example, diabetes, HI/HA, or other insulin-related disorders. As shown herein, the regulation of GDH activity is strongly correlated with the regulation of insulin secretion. Accordingly, as described herein, the invention provides a new target implicated in insulin secretion for pharmacologic agents, whether currently existing or developed hereafter, to block, interact with, inhibit, bind to, or otherwise regulate. In addition, the invention provides compositions, e.g., EGCG and ECG, and methods that may be used to inhibit GDH activity, thereby allowing an investigator to research the effects of other agents on other enzymes or cellular components involved in insulin secretion.

The following examples are provided to further illustrate the compositions and methods of the present invention. These examples are illustrative only and are not intended to limit the scope of the invention in any way.

### EXAMPLES

### Example 1

In this Example, the effects of epigallocatechin gallate (EGCG), epigallocatechin (EGO), epicatechin gallate (ECG), and epicatechin (EC) from Camellia sinensis on GDH activity were tested. Bovine GDH (bGDH) was used in this Example, which was obtained as an aqueous (NH₄)₂SO₄ suspension from Sigma Aldrich Chemical Company (St. Louis, Mo.).

First, aliquots of GDH were extensively dialyzed against 0.1 M sodium phosphate buffer, pH 7.0, which contained 1 mM ethylenediaminetetraacetic acid (EDTA). The enzyme concentrations were adjusted to 1 mg/ml and the amount of enzyme added to the reaction mixture, for kinetic analysis, was adjusted to yield optimal steady state velocity measurements. All solutions were made immediately prior to use. Enzyme assays were performed by monitoring reduced coenzyme absorbance at 340 nm using a Shimadzu UV-1601 spectrophotometer. The reductive amination reactions were performed in 3 mL volumes at pH 7.0 in the presence of 0.1 mM nicotinamide adenine dinucleotide (NADH), 50 mM NH₄Cl, and 5 mM 2-oxoglutarate. The rate of NADH oxidation was calculated using an extinction coefficient of 6.22 mM⁻¹cm⁻¹.

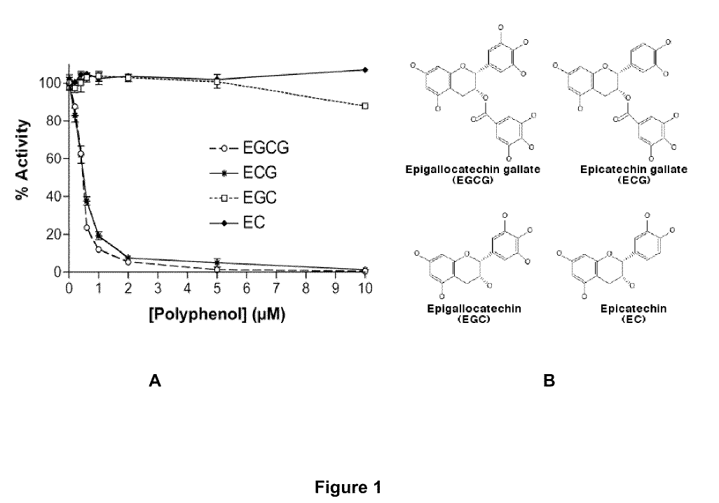

As shown in FIG. 1A, EGCG and ECG, but not EGC and EC, are potent inhibitors of GDH activity with ED₅₀ values of approximately 300 nM. Since all four polyphenols (EGCG, ECG, EGC, and EC) have comparable antioxidant activities, such data strongly suggest that EGCG and ECG effects are allosteric in nature. In addition, such inhibition was shown to be reversible, as dialysis of an EGCG/GDH mixture completely alleviated the inhibition (data not shown).

### Example 2

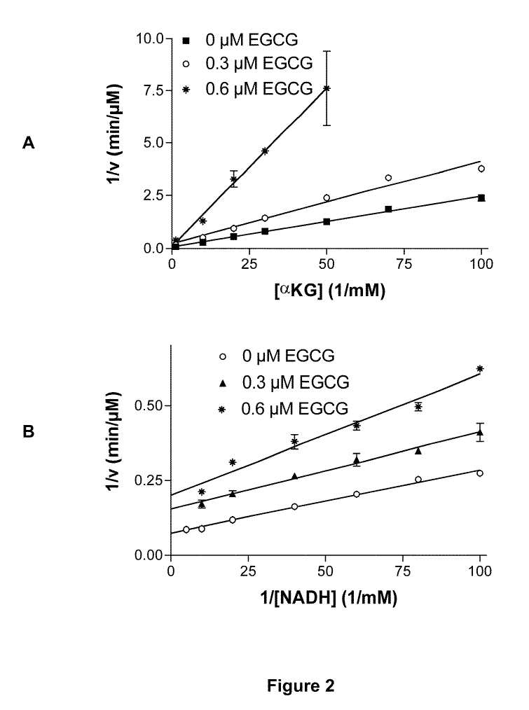

In this Example, EGCG was added to a reaction (at a final concentration of 0.0, 0.3, or 0.6 μM), along with various concentrations of NADH or 2-oxoglutarate, to further ascertain how EGCG inhibits the reductive amination reaction catalyzed by GDH. Such kinetic analysis was carried out as described above in Example 1. The results of such analysis were examined using Lineweaver-Burke plots, shown in FIG. 2. As summarized in Table 1 below (and FIG. 2), EGCG affects both the slope and Y-intercept of the curves in such Lineweaver-Burke plots in a manner consistent with non-competitive inhibition.

TABLE 1

| | NADH varied | | | 2-oxoglutarate varied | | |
|---|---|---|---|---|---|---|
| [EGCG] | 0 μM | 0.3 μM | 0.6 μM | 0 μM | 0.3 μM | 0.6 μM |
| Slope | 0.0021 ± 0.00007 | 0.0026 ± 0.0002 | 0.0040 ± 0.0003 | 0.024 ± 0.0007 | 0.039 ± 0.002 | 0.15 ± 0.02 |
| Y-intercept | 0.075 ± 0.004 | 0.15 ± 0.01 | 0.2000 ± 0.02 | 0.095 ± 0.04 | 0.24 ± 0.1 | 0.097 ± 0.5 |
| R² | 0.9880 | 0.9504 | 0.9583 | 0.9896 | 0.9701 | 0.9034 |

That is, the above data suggest that EGCG does not act by directly competing with either coenzyme or substrate binding to the active site. Rather, the data suggest that EGCG inhibits GDH activity by binding to either the free enzyme or the enzyme-substrate complex.

### Example 3

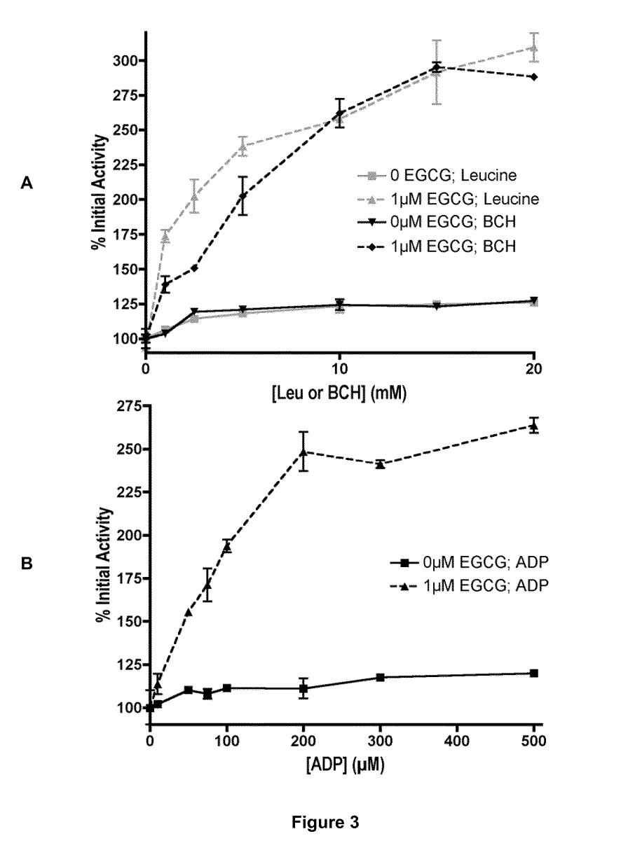

The ability of the activators BCH, leucine, and ADP to reverse EGCG regulation on GDH was next examined. More particularly, kinetic analysis of GDH activity was carried out as described in Example 1, wherein each reaction contained either (a) 0 μM EGCG plus leucine; (b) 1 μM EGCG plus leucine; (c) 0 μM EGCG plus BCH; (d) 1 μM EGCG plus BCH; (e) 0 μM EGCG plus ADP; or (f) 1 μM EGCG plus ADP. The concentrations of leucine, BCH, and ADP in this Example were varied, as shown in FIG. 3.

As shown in FIG. 3, under these assay conditions, all three activators only increased the velocity of the reaction by approximately 30% in the absence of EGCG. However, in the presence of EGCG, all three regulators activated the reaction by nearly three-fold. In all three cases, the addition of 1 μM EGCG inhibited the reaction by more than 80% and the highest concentration of activators decreased EGCG inhibition to ˜30%. It should be noted that previous studies have shown that ADP and leucine activate GDH by binding to spatially different sites. Abrogation of EGCG inhibition by these allosteric activators further demonstrates that EGCG acts in an allosteric manner.

### Example 4

As described herein, HI/HA syndrome is caused by impaired (or abrogated) GDH sensitivity to GTP inhibition. Many of the mutations present in the GDH enzyme of HI/HA individuals reside in the GTP binding site, and thus, likely act by sterically interfering with GTP binding. Accordingly, the invention provides that EGCG and ECG, compositions that contain EGCG and/or ECG (such as Camellia sinensis extract), and other compounds and compositions that specifically inhibit GDH activity are therapeutically useful if such compositions also inhibit the HI/HA mutant forms of GDH.

To this end, EGCG was tested against five different HI/HA mutant forms of GDH, namely, R265K, R269C, E296A, S448P, and H454Y. In addition, EGCG was tested against human wild type GDH (“WT”), GDH from Tetrahymena thermophilia (“tGDH”) that, like the HI/HA mutant GDH enzymes, is not regulated by GTP, and a human GDH that was missing a certain 48 residue ‘antenna-like’ feature protruding from the top of each of its six subunits that is necessary for ADP, GTP, and Palmitoyl CoA regulation. Such WT, ‘antenna-less’, and HI/HA GDH enzymes were expressed in and purified from E. coli, whereas tGDH was purified from Tetrahymena thermophilia. The kinetic analysis of such GDH enzymes was carried out as described in Example 1 above.

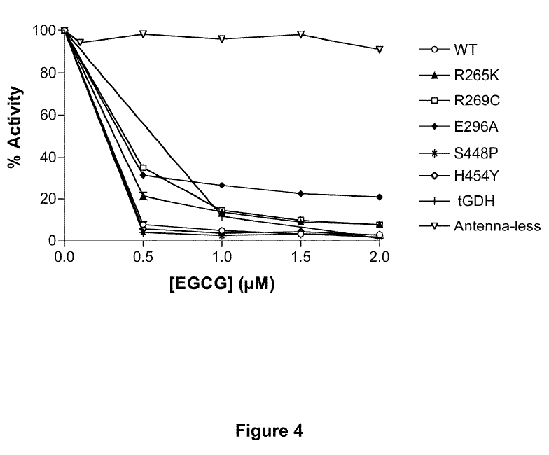

Referring to FIG. 4, EGCG inhibited the activity of HI/HA GDH enzymes with substantially the same efficacy as with wild type human GDH. In addition, EGCG inhibited GDH from Tetrahymena thermophilia. These results suggest that EGCG acts independent of the GTP inhibitory site. Interestingly, EGCG did not inhibit the ‘antenna-less’ form of human GDH. Such data show that EGCG inhibition of GDH activity is unrelated to its antioxidant activity, is independent of the GTP inhibitory site, does not directly affect the active site of GDH, and may be dependent upon the antenna-like structure present in GDH to exert its activity.

### Example 5

Since EGCG and ECG were found to be potent inhibitors of GDH in vitro, it was postulated that GDH-dependent β-cell functions should also be influenced by these catechins. For example, the phenomenon of leucine stimulated insulin secretion (“LSIS”) has been recently shown to be mediated by GDH and its regulation of glutamineolysis. LSIS is only observed after a prolonged period of “run-down” to produce a state of energy depletion. Under these conditions, the levels of GDH inhibitors, ATP and GTP, are reduced while the concentration of the GDH activator, ADP, is increased. When leucine (or its non-metabolizable analog, beta-2-aminobicyclo[2.2.1]heptane-2-carboxylic acid, “BCH”) is then added to cells in this depleted state, the flux of glutamine through glutaminase and GDH is increased, ATP is generated, and the β-cells are stimulated to secrete insulin.

In this Example, adult male Wistar rat islets were isolated by collagenase digestion and initially cultured in glucose-free RPMI 1640 medium (Sigma Aldrich Chemical Company, St. Louis, Mo.). The culture medium was supplemented with 10% fetal bovine serum, 2 mM glutamine, 100 units/mL penicillin, and 50 μg/mL streptomycin.

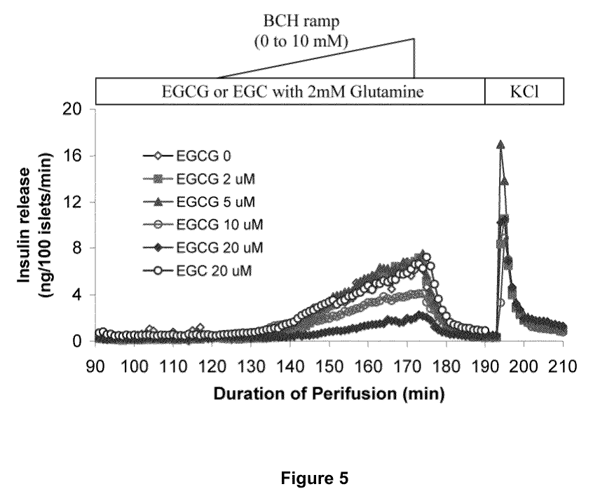

The islets were then perifused in the absence of glucose and in the presence of 2 mM glutamine (and the different concentrations of EGCG or EGC shown in FIG. 5) for run-down periods of 120 minutes prior to stimulation with a BCH ramp (0 to 10 mM, 0.2 mM/minute). Specifically, 100 cultured rat islets were loaded onto nylon filters in a small chamber and perifused in a Krebs-Ringer bicarbonate buffer (115 mmol/liter NaCl, 24 mmol/liter NaHCO₃, 5 mmol/liter KCl, 1 mmol/liter MgCl₂, 2.5 mmol/liter CaCl₂, in 10 mM HEPES, pH 7.4) with 0.25% bovine serum albumin at a flow rate of 2 mL/minute. Perifusate solutions were gassed with 95% O₂/5% CO₂ and maintained at 37° C. Finally, the islets were exposed to 30 mM KCl. Samples were collected every minute to measure insulin levels by radioimmunoassay.

As shown in FIG. 5, the BCH stimulation of insulin secretion was blocked by EGCG, but not EGO, in a dose-dependent manner with an ED₅₀ of less than 10 μM. EGCG did not affect the intrinsic ability of the cells to secrete insulin, since depolarization by KCl still resulted in release of insulin. The concentration of EGCG required to abrogate LSIS, however, was significantly higher than what was shown to be necessary to abrogate GDH activity in-vitro. This is likely due to the bioavailability of EGCG in the mitochondria and/or that higher levels of EGCG are required in tissue to overcome antagonism by leucine, ADP, and BCH.

### Example 6

In this Example, the effects of EGCG and EGO on LSIS were measured, while simultaneously monitoring respiration rates. More particularly, the effects of EGCG (20 μM) and EGO (20 μM) on BCH-stimulated insulin secretion were measured. First, 1000 adult male Wistar rat islets were isolated as described above and were cultured on a glass perifusion chamber in 10 mM glucose for 3 days. Next, the islets were perifused in an oxygen consumption measurement apparatus containing BCH, along with 0 μM EGCG and 0 μM EGO, 20 μM EGCG, or 20 μM EGO.

The perifusion apparatus used in these Examples consisted of a peristaltic pump, a water bath (37° C.), a gas exchanger (artificial lung: media flowed through the thin-walled silastic tubing loosely coiled in a glass jar that contained 20% O₂ and 5% CO₂ balanced with N₂), and fraction collector. All transfer lines were insulated.

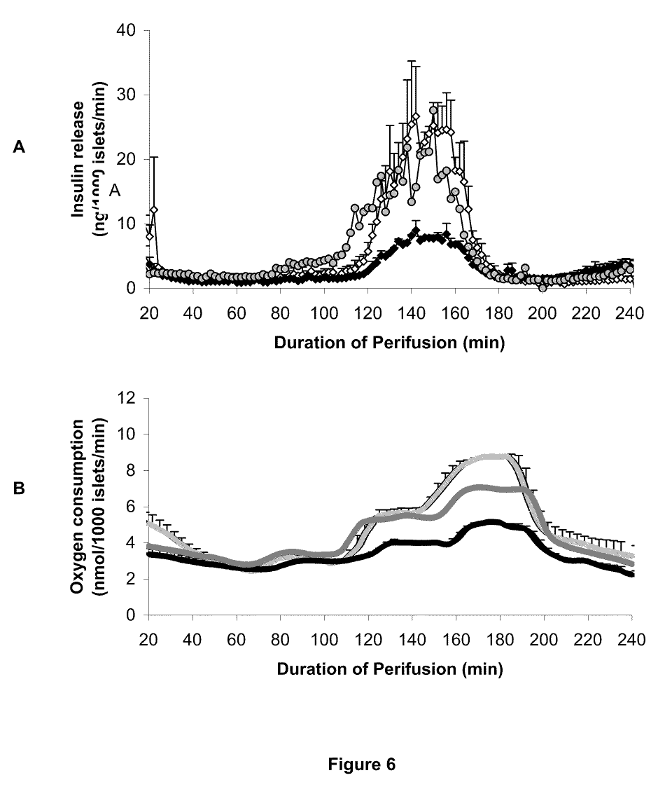

After the system was provided with 20 μM EGCG or 20 μM EGO, oxygen partial pressure was recorded every 10 seconds by phosphorescence lifetimes of an oxygen-sensitive porphyrin (palladium-mesotetra; 4-carboxyphenyl porphyrin dendrimer). The dye molecules were excited with pulses at 524 nm from a UV lead and emission was measured at 690 nm. The inflow oxygen tension was measured in the absence of islets in the chamber before and after each experiment. The perifusate was a Krebs buffer (pH 7.4) containing 2.2 mM Ca²⁺, 1% bovine serum albumin equilibrated with 20% O₂ and 5% CO₂ balanced with N₂. The flow rate was 100 mL/min and samples were collected every 2 minutes for insulin measurements by radioimmunoassay. The results are summarized in FIG. 6, and are presented as means±S.E. for 1000 islets from 3 separate experiments for EGCG (and from 1 experiment for EGO). Due to the high density of data, S.E. is only shown in every 20 samples.

As shown in FIG. 6A, 20 μM EGCG strongly inhibited LSIS (i.e., BCH-induced insulin secretion) (which is represented therein by the solid diamonds). Similarly, as shown in FIG. 6B, 20 μM EGCG inhibited the BCH-induced increase in respiration rates (which is represented therein by the black line). The addition of EGCG itself, however, did not have any significant effect on oxygen consumption in the absence of BCH (data not shown).

EGO exhibited a number of interesting effects on LSIS. For example, it did not cause significant inhibition of LSIS (FIG. 6A; represented as grey circles), nor did it have as strong of an effect on respiration as compared to EGCG (FIG. 6B; EGO is represented therein by the dark grey line). In addition, EGO seemed to cause a slight sensitization of the β-cells to BCH as manifested in a slightly faster response to BCH-mediated insulin secretion and respiration enhancement. Such data clearly demonstrate that the EGCG effects on GDH activity described herein are not due to its antioxidant activity and, furthermore, that EGO may have some, albeit quite different, effects on the pancreatic cells.

### Example 7

In this Example, the effects of EGCG and DON (6-diazo-5-oxo-L-norleucine) on BCH-stimulated glutamine oxidation were measured.

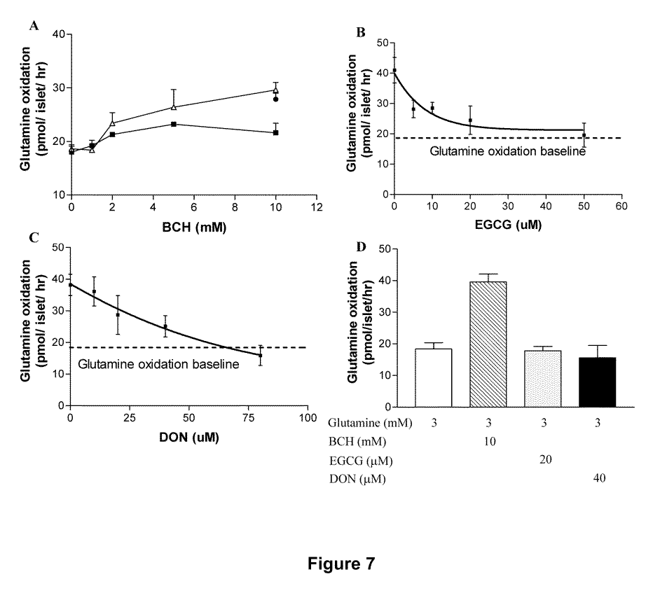

In a first experiment, a batch of 100 islets were incubated with glucose-free Krebs-Ringer bicarbonate buffer, as described herein, containing 20 μM EGCG or 20 μM EGO and 2 mM glutamine for 60 minutes. The islets were then incubated in the presence of varying concentrations of BCH (shown in FIG. 7A) for 60 minutes, and for an additional 60 minutes in the presence of 2 mCi [U-¹⁴C]-glutamine (NEN-Life Science Products, UK).

Next, [U-¹⁴C]-glutamine oxidation in the incubated islets was measured. A trap filter was placed in each tightly sealed glass tube of the perifusion apparatus to collect the ¹⁴CO₂ produced by the islets, and the amount of radioactivity was determined by liquid scintillation counting. The results are summarized in FIG. 7A, and are presented as means±S.E. from 4 separate experiments. As shown in FIG. 7A, EGCG (represented by solid squares), but not EGO (represented by the solid circle), completely blocked BCH stimulation of glutaminolysis (glutamine oxidation).

In a second experiment, a batch of 100 islets were incubated with glucose-free Krebs-Ringer bicarbonate buffer, as described herein, containing varying concentrations of EGCG or DON (as shown in FIGS. 7B and 7C, respectively) and 3 mM glutamine for 60 minutes. The islets were then incubated in the presence of 10 mM BCH (or no BCH—to measure “baseline” glutamine oxidation levels) for 60 minutes. The islets were then incubated for an additional 60 minutes in the presence of 2 mCi [U-¹⁴C]-glutamine (NEN-Life Science Products, UK). Next, [U-¹⁴C]-glutamine oxidation in the incubated islets was measured, as described above.

Referring to FIG. 7B, the data suggest that EGCG inhibits glutamine oxidation in a dose-dependent manner, with maximal effects occurring at approximately 20 μM (and never decreasing glutamine oxidation below that of the cells not stimulated by BCH). As shown in FIG. 7C, the effects of EGCG on glutamine oxidation is in contrast to the effects of DON, an inhibitor of glutaminase, which blocks glutaminolysis to levels lower than the unstimulated β-cells at high concentrations. The foregoing results are summarized in FIG. 7D, which further demonstrate that EGCG does not have an effect on basal levels of glutamine oxidation, but does block BCH (i.e., leucine) enhancement of glutaminolysis. In light of the kinetic analysis described above and this Example 7, it is clear that EGCG effects on LSIS are due to inhibition of GDH activity.

In the case of LSIS (and the BCH-induced model described above), the β-cell is substantially depleted of glucose and provided with glutamine at low concentrations prior to the application of leucine (i.e., BCH). Therefore, when leucine (i.e., BCH) is applied, the lack of ATP/GTP inhibition combined with ADP and BCH/leucine activation facilitates glutaminolysis. This leads to the generation of ATP and, combined with the exogenously added glutamine acting as an intracellular signal, insulin secretion is facilitated. Under these conditions, the primary force driving ATP production and subsequent insulin release is enhanced amino acid oxidation with BCH/leucine stimulating this process. When EGCG is introduced, EGCG inhibits GDH activity, blocks glutaminolysis, and thereby prevents the BCH/leucine effects.

### Example 8

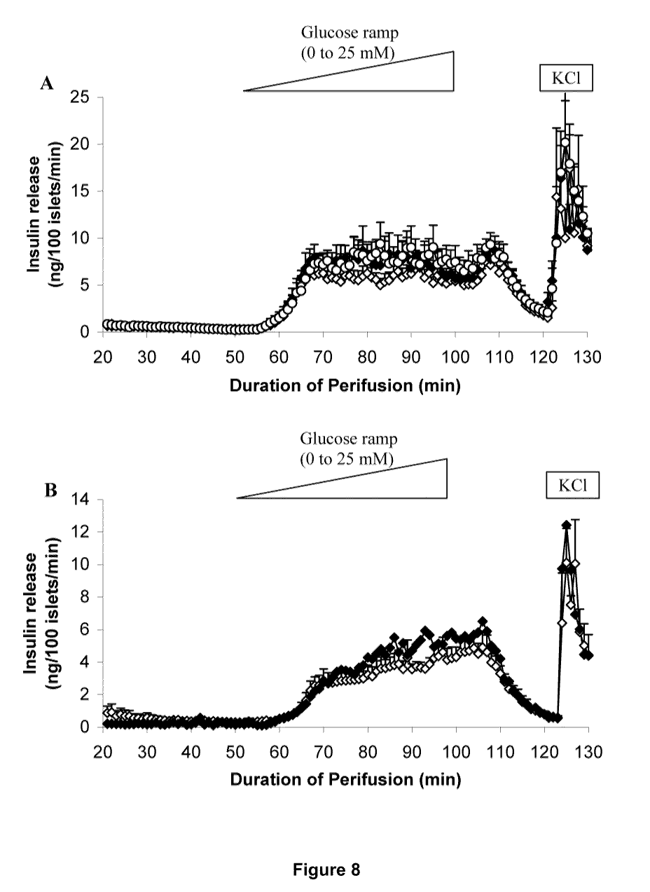

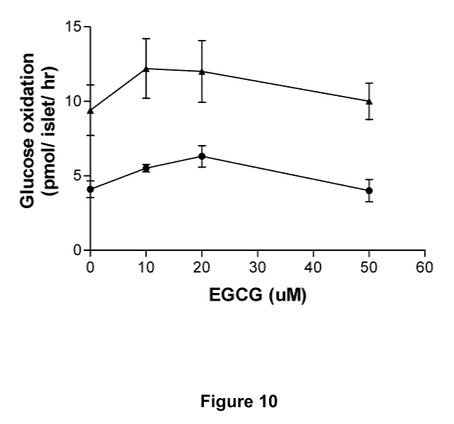

In this Example, the effect of EGCG on glucose stimulated insulin secretion (GSIS) was examined. Isolated rat islets were cultured with 20 mM glucose (FIG. 8A) and 10 mM glucose (FIG. 8B) for 3 days, then perifused in the absence of glucose for 50 minutes and in the presence of 0 μM EGCG; 20 μM EGCG; or 20 μM EGC. Next, the islets were stimulated by a glucose ramp (0 to 25 mM, 0.5 mM/minute). Finally, the islets were exposed to 30 mM KCl. Insulin levels were measured every minute by radioimmunoassay. The results shown in FIG. 8A are presented as means±S.E. for 100 islets from 3 separate experiments (and means±S.E. for 100 islets from 1 experiment for FIG. 8B). As shown in FIGS. 8A and 8B, in all cases, neither EGO nor EGCG affected GSIS under these short, run-down conditions.

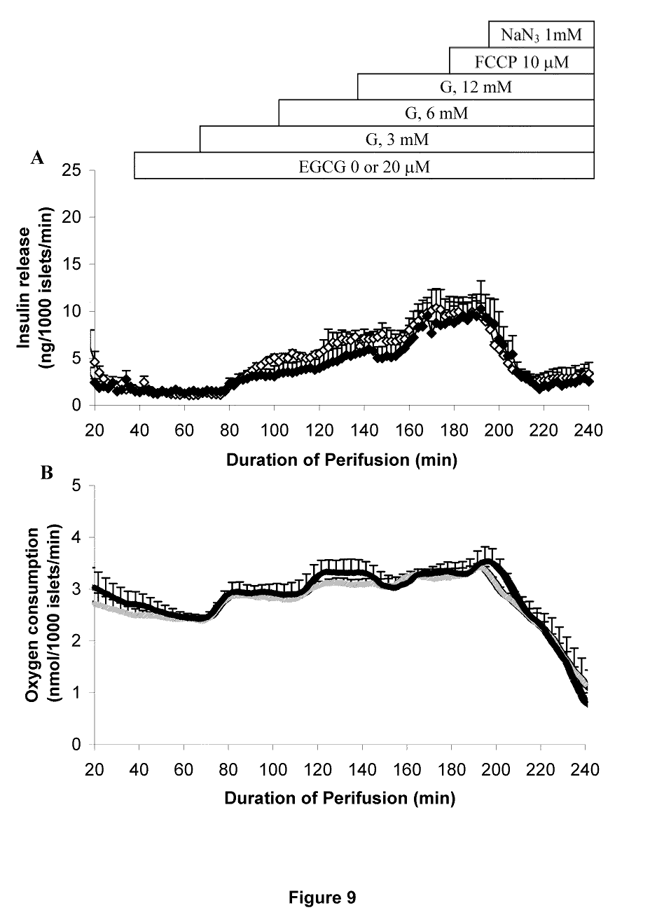

Next, insulin secretion and oxygen consumption were measured, as described herein, during glucose stimulation to confirm the above results. Isolated rat islets were isolated and cultured, as described above, with 10 mM glucose for 3 days and subsequently perifused in the oxygen consumption measurement apparatus described above. EGCG, glucose (G), Mesoxalonitrile 4-trifluoromethoxyphenylhydrazone (FCCP), and NaN₃ were added in the sequence and amounts shown in FIG. 9. In general, FCCP is known to stimulate Mg²⁺-ATPase activity, inhibit β-amyloid production, and mimic the effect of selective glutamate agonist N-methyl-D-aspartate (NMDA) on mitochondrial superoxide production. The results shown in FIG. 9 are presented as means±S.E. for 1000 islets from 3 separate experiments. Due to the high density of data, S.E. is only shown in every 20 samples.

Referring to FIGS. 9A and 9B, the results suggest that EGCG does not affect GSIS or respiration under these conditions.

In addition, the effect of EGCG on glucose oxidation was measured. More particularly, batches of 100 islets were prepared as described herein and preincubated with glucose-free Krebs-Ringer bicarbonate buffer containing varying concentrations of EGCG (shown in FIG. 10) for 60 minutes. Islets were then incubated with 3 mM or 12 mM glucose, in the presence of the varying concentrations of EGCG, for another 60 minutes, along with 2 mCi [U-¹⁴C]-glucose (NEN-Life Science Products, UK). Production of ¹⁴CO₂ was monitored as described above.

As shown in FIG. 10, EGCG did not affect glucose oxidation over a wide range of EGCG concentrations when the cells were incubated in either 3 mM or 12 mM glucose.

In light of the foregoing, EGCG and ECG, in and of themselves, do not appear to affect glucose-mediated respiration, insulin secretion, or glucose oxidation. Rather, the invention provides that EGCG necessarily affects insulin secretion via modulation of GDH activity. In the case of GSIS, the effects of EGCG are likely dependent upon the energy state of the cell. That is, under the brief run-down conditions used for the GSIS analysis described herein, for example, glutaminolysis was not the main energy source for the cells and the levels of high-energy metabolites (e.g., GTP and ATP) were not fully depleted. As a result, such metabolites are still present at sufficient levels to effectively inhibit GDH activity.

### Example 9

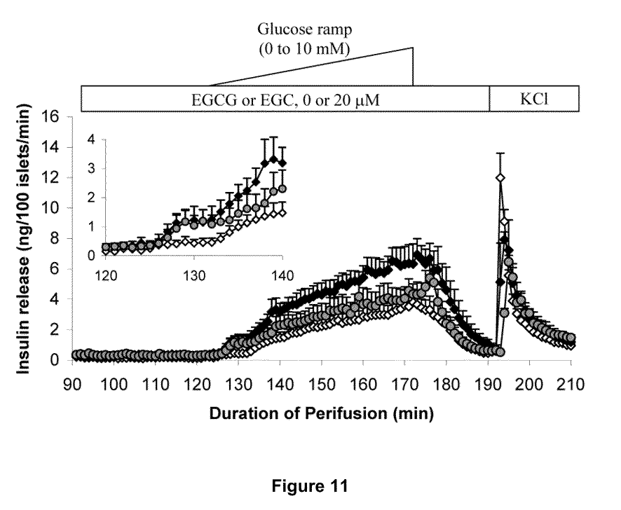

Under brief “run-down” conditions used for the above GSIS analysis, where glucose was removed from the cells for only 50 minutes, the levels of high-energy metabolites (i.e., GTP and ATP) have not been depleted and these effectively shut down GDH activity. This, in turn, eliminated any effect GDH might have on insulin secretion. Therefore, the effects of EGCG were also tested on β-cells that had been “run-down” for a longer period of time (120 minutes) prior to glucose stimulation (FIG. 11). Specifically, isolated rat islets were cultured with 10 mM glucose for 3 days and then perifused in the absence of glucose and in the presence of 0 or 20 μM EGCG (or 20 μM EGO) for “run-down” periods of 120 minutes prior to stimulation with a glucose ramp (0 to 25 mM, 0.5 mM/minute). Finally, islets were exposed to 30 mM KCl. The insert highlighted the insulin secretion from time=120 minutes to 140 minutes.

Under these conditions, with low ATP and GTP concentrations and high ADP concentrations, GDH inhibition was relieved and the enzyme was able to influence insulin secretion. Here, in contrast to the short “run-down” conditions described in the previous Example, EGCG, but not EGO, potentiates GSIS. As before, neither EGCG or EGO affected KCl-stimulated release of insulin. EGO was shown to be inactive compared to EGCG, but seems to slightly sensitize the cells at low glucose concentrations (FIG. 11, inset).

In light of the foregoing, in the case of GSIS, the effects of EGCG are dependent upon the energy state of the cell. When the β-cell has been “run-down” for a brief period of time, glutaminolysis is not the main energy source for the cell and the high-energy metabolites (e.g., GTP and ATP) are still present and strongly inhibit GDH activity. Under these conditions, EGCG does not affect GSIS since GDH is already effectively inhibited.

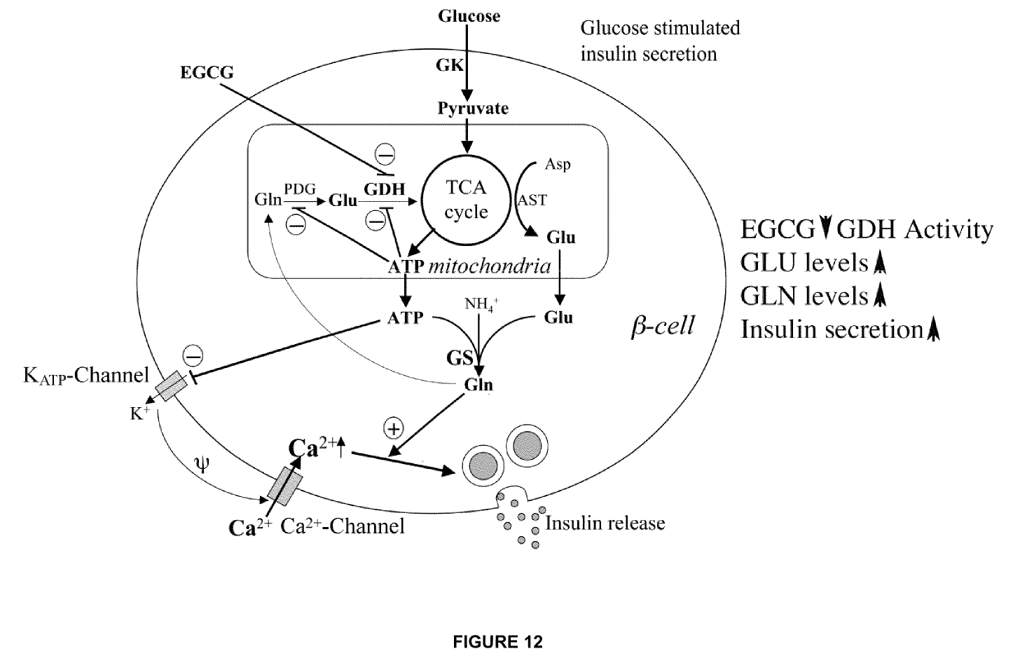

However, when the cell has been “run-down” for longer periods of time, the high-energy inhibitors, ATP and GTP, have been depleted and the levels of the activator, ADP, have been increased. This is summarized in the model shown in FIG. 12. Under these conditions, GDH has been released from its inhibition and can significantly affect the intracellular glutamine pool. As glucose is added, without glutamine being exogenously provided, the cytoplasmic pool of glutamine is limited while the supply of ATP is not. Therefore, when EGCG is added, the inhibition of GDH causes an increase of the glutamine pool and glucose stimulation of insulin secretion is potentiated. Therefore, the effect of EGCG on GDH activity was shown to be the same in both LSIS and GSIS under long “run-down” conditions, but the role of GDH has changed; from being used to supply the cell with energy in the case of LSIS to being used to regulate a limited pool of cellular glutamine in the case of GSIS. Therefore, this Example further suggests that GDH is a novel target for the treatment of disorders relating to the dysfunctional regulation of insulin secretion, such as type II diabetes, and, furthermore, that EGCG may be useful as an inhibitor of GDH activity.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for modulating the activity of glutamate dehydrogenase comprising providing to a system in need thereof with an effective amount of a compound selected from the group consisting of EGCG and ECG, including salts, hydrates, solvates, N-oxides, and combinations thereof.

2. The method of claim 1, wherein the compound is EGCG, including salts, hydrates, solvates, and N-oxides thereof.

3. The method of claim 2, wherein EGCG is represented by the following structure:

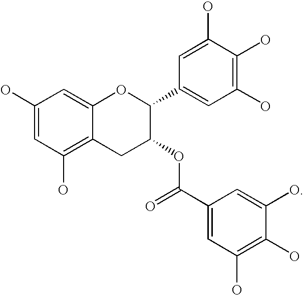

4. The method of claim 3, wherein EGCG is in a substantially isolated and purified form.

5. The method of claim 1, wherein the compound is ECG, including salts, hydrates, solvates, N-oxides, and combinations thereof.

6. The method of claim 5, wherein ECG is represented by the following structure:

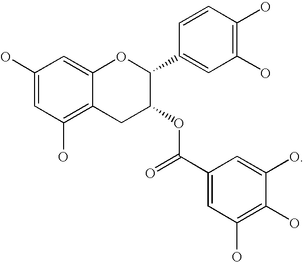

7. The method of claim 6, wherein ECG is in a substantially isolated and purified form.

8. The method according to claim 1, wherein the providing step comprises administering the compound to an in vitro system.

9. The method according to claim 8, wherein the in vitro system comprises purified glutamate dehydrogenase.

10. The method according to claim 1, wherein the providing step comprises administering the compound to a human.

11. The method according to claim 1, wherein the compound inhibits or reduces the activity of glutamate dehydrogenase.

12. The method according to claim 11, wherein the compound allosterically inhibits or reduces the activity of glutamate dehydrogenase.

13. A method for regulating insulin secretion comprising providing to a patient in need thereof with an effective amount of a composition selected from the group consisting of Camellia sinensis extract, EGCG, and ECG, including salts, hydrates, solvates, N-oxides, and combinations thereof.

14. The method according to claim 13, wherein the composition is EGCG, which is represented by the following structure: 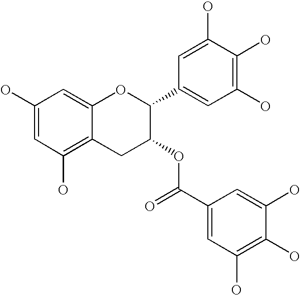
, including salts, hydrates, solvates, and N-oxides thereof.

15. The method according to claim 13, wherein the composition is ECG, which is represented by the following structure: 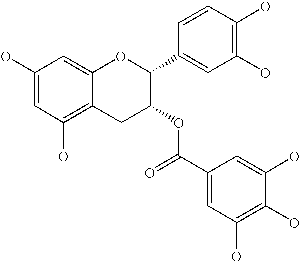
, including salts, hydrates, solvates, and N-oxides thereof.

16. The method according to claim 13, wherein the composition is provided to a patient to regulate leucine stimulated insulin secretion.

17. A method for treating or preventing the effects of HI/HA comprising providing to a patient in need thereof with an effective amount of a compound selected from the group consisting of EGCG and ECG, including salts, hydrates, solvates, N-oxides, and combinations thereof.

18. The method according to claim 17, wherein the compound is represented by the following structure: 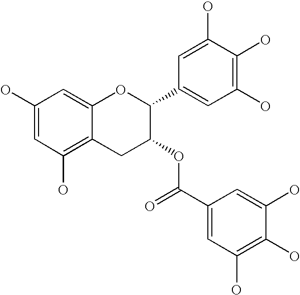
, including salts, hydrates, solvates, and N-oxides thereof.

19. The method according to claim 17, wherein the compound is represented by the following structure: 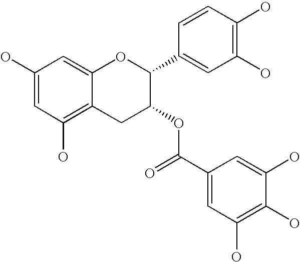
, including salts, hydrates, solvates, and N-oxides thereof.
